(19) United States
(12) Patent Application Publication
(10) Pub. No.: US 2012/0135882 A1
(43) Pub. Date: May 31, 2012

(54) METHODS FOR DIAGNOSING CHRONIC KIDNEY DISEASE AND ASSESSING THE RISK OF DISEASE PROGRESSION

(75) Inventor: Erwin P. Bottinger, New York, NY (US)

(73) Assignee: MOUNT SINAI SCHOOL OF MEDICINE, New York, NY (US)

(21) Appl. No.: 13/320,485

(22) PCT Filed: May 13, 2010
(86) PCT No.: PCT/US10/34726

§ 371 Date: Jan. 31, 2012

Related U.S. Application Data
(60) Provisional application No. 61/178,388, filed on May 14, 2009.

(51) Int. Cl. C40B 30/04; G01N 33/567; C40B 40/06; C12Q 1/68

(52) U.S. Cl. 506/9; 435/6.12; 435/7.21; 506/16; 435/6.11

(57) ABSTRACT

Methods and compositions for diagnosing chronic kidney disease and assessing the risk of disease progression, as well as methods of screening for molecular biomarkers useful for diagnosing the likelihood of disease progression, are provided.

This invention was made with U.S. government support under grant numbers 5R01DK060043, 5R01DK073960, 5U01DK060995, and R21DK079441 awarded by the National Institutes of Health. The government has certain rights in the invention.

### FIELD OF THE INVENTION

The present disclosure relates to methods related to diagnosing chronic kidney disease and assessing the risk of disease progression, as well as methods of screening for molecular biomarkers useful for diagnosing the likelihood of disease progression.

### BACKGROUND OF THE INVENTION

Awareness of chronic kidney disease (CKD) and its consequences has increased enormously during the past decade as a result of the worldwide adoption of a uniform classification system developed by the Kidney Disease Outcomes Quality Initiative (K/DOQI). (Am. J. Kidney Dis., 2002, 39:S1-S266). Recent epidemiological studies indicate that 16.8% of the US population may be affected with CKD (MMWR Morb. Mortal. Wkly. Rep. 2007, 56:161-165) suggesting that CKD is far more prevalent in the general population than previously thought. Importantly, cardiovascular disease morbidity and mortality are strongly associated with advanced CKD and proteinuria before end-stage renal disease, revealing that some underlying pathomechanisms may be shared, or interactive, between cardiovascular disease and CKD (Freedman et al.,: Hypertension, 2006, 48:8-13; Garg and Bakris, Vasc. Med., 2002, 7:35-43; Schieppati and Remuzzi, Kidney Int. Suppl., 2005, 98:S7-S10).

Progression of CKD to end-stage renal disease occurs only in a minority of CKD patients, indicating considerable heterogeneity in the risk of progressive decline of renal function in CKD. Because the Kidney Disease Outcomes Quality Initiative (K/DOQI) of the National Kidney Foundation definition of CKD stages is based on glomerular filtration rate (GFR) alone, the relative risk of progression of patients within each stage is not characterized. As a simple method of risk assessment, family history of advanced CKD or end-stage renal disease and the extent of proteinuria are currently the best predictors of the risk to develop progressive CKD. (Satko et al., Semin. Dial., 2007, 20:229-236; and Taal and Brenner, Kidney Int., 2008, 73:1216-1219). However, the accuracy of these clinical markers is currently not sufficient to reliably predict the risk of CKD progression, or to guide preventive interventions.

Thus, one of the most important unmet needs in renal medicine is the identification and validation of predictive markers of CKD progression that facilitate targeted treatment of those at high risk, while avoiding unnecessary treatment and the attendant financial costs in low-risk patients (Taal and Brenner, Kidney Int., 2006, 70:1694-1705) However, in contrast with other disorders that are characterized by heterogeneity in progression, in particular malignancies (Beer et al., Nat. Med., 2002, 8:816-824 and Ye et al., Nat. Med., 2003, 9:416-423) the development of predictive molecular markers in CKD is limited by the scarcity of tissue-based diagnostic procedures in clinical renal medicine.

During the past decade, the application of gene expression profiling in cancer research has resulted in development of new therapeutic targets, and of prognostic profiling assays that are now in phase III clinical trials designed to evaluate their contribution to therapeutic decision making (Morris and Carey, Curr. Opin. Oncol. 2007, 19:547-551, incorporated herein by reference). The impressive progress and successes of genomic profiling in oncology have been facilitated by an abundance of surgical tumor tissues and samples from hematological malignancies. In contrast with the success of gene expression profiling in oncology, several challenges have severely limited the application of genomic profiling in nonmalignant kidney diseases. First, tissue availability is limited because diagnostic kidney biopsies or nonmalignant nephrectomies are performed relatively infrequently. Second, the composition of kidney tissue cores is inherently heterogeneous contributing to sampling error (Corwin et al., Am. J. Nephrol., 1988, 8:85-89) which renders standardized, quantitative gene expression profiling across large series of kidney biopsies technically challenging (Yasuda et al., Clin. Exp. Nephrol., 2006, 10:91-98).

Long-term data on progression of CKD demonstrate that the high incidence of end-stage renal disease (ESRD) in patients on “optimal” therapy remains unacceptable. In contrast, stage III or higher CKD does not progress in a fraction (26% to 34%) of CKD patients. Thus, development of “predictive” biomarkers that characterize the risk of CKD progression in individual patients is an important unmet need.

### SUMMARY OF THE INVENTION

Methods and compositions for assessing the risk of CKD progression are described. With respect to the number of molecular biomarkers used in the methods described herein, “plurality” generally refers to at least 3, 4, 5, 6, 7, 8, 9, 10, 11, 12, 13, 14, 15, 16, 17, 18, 19, 20, 21, 22, 23, 24, or 25 biomarkers. The methods provided herein also contemplate the use of a single (i.e., one) molecular biomarker. It is also contemplated that polynucleotide-based molecular biomarkers include all forms of RNA (e.g., mRNA, tRNA, rRNA) and DNA (e.g., genes and fragments thereof, cDNA).

In one embodiment of the invention, a method of assessing the risk of chronic kidney disease (CKD) progression is provided comprising the steps of: a) obtaining a biological sample from a subject; and b) measuring in the sample the expression level of a plurality of molecular biomarkers selected from the group consisting of those biomarkers identified in Table 5, wherein the biomarkers include the identified mRNA transcription products of the genes identified in Table 5 and proteins encoded thereby; and c) comparing the expression profile of the molecular biomarkers to a control expression profile, thereby assessing the risk of CKD progression. In specific embodiments, the aforementioned method is provided wherein the biological sample is a kidney tissue sample or urine sample. In another specific embodiment, the aforementioned method is provided wherein the subject is human. In still another specific embodiment, the aforementioned is provided wherein the expression level of the mRNA transcription products is measured by contacting the biological sample with nucleic acid probes under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, thereby allowing the formation of a hybrid complex between the mRNA transcription products and the nucleic acid probes; and detecting the formation of the complex. In still another specific embodiment, the aforementioned method is provided wherein the level of protein expression is measured using antibodies, wherein at least one antibody is capable of specifically binding to each of the proteins. In another specific embodiment, the aforementioned method is provided wherein the expression of at least one molecular biomarker in the sample is increased relative to the control and the expression of at least one other molecular biomarker in the sample is decreased, relative to the control. In yet another specific embodiment, the aforementioned method is provided wherein a 2- to 10-fold increase or decrease in the amount of expression of the plurality of molecular biomarkers complex compared to a control is indicative of a likelihood of CKD progression. In a related embodiment, the increase or decrease in the amount of expression is 2- to 3, 4, 5, 6, 7, 8, 9, 10, 20, 30, 40, 50, 60, 70, 80, 90 or 100-fold.

In another embodiment of the invention, a method of screening for a molecular biomarker useful in assessing the risk of CKD progression is provided comprising the steps of: a) obtaining a biological sample from a subject suffering from CKD or a disease associated with CKD; b) measuring expression level of a candidate molecular biomarker in the sample; and c) comparing the expression profile of the candidate molecular biomarker to a control expression profile of the candidate molecular biomarker, thereby identifying a candidate molecular biomarker as a molecular biomarker useful in assessing the risk of CKD progression. In specific embodiments, the aforementioned method is provided wherein the biological sample is a kidney tissue sample or urine sample. In another specific embodiment, the aforementioned method is provided wherein the subject is human. In still another specific embodiment, the aforementioned is provided wherein the expression level of the mRNA transcription products is measured by contacting the biological sample with nucleic acid probes under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, thereby allowing the formation of a hybrid complex between the mRNA transcription products and the nucleic acid probes; and detecting the formation of the complex. In still another specific embodiment, the aforementioned method is provided wherein the level of protein expression is measured using antibodies, wherein at least one antibody is capable of specifically binding to each of the proteins. In another specific embodiment, the aforementioned method is provided wherein the expression of at least one molecular biomarker in the sample is increased relative to the control and the expression of at least one other molecular biomarker in the sample is decreased, relative to the control. In yet another specific embodiment, the aforementioned method is provided wherein a 2- to 10-fold increase or decrease in the amount of expression of the plurality of molecular biomarkers complex compared to a control is indicative of a likelihood of CKD progression. In a related embodiment, the increase or decrease in the amount of expression is 2- to 3, 4, 5, 6, 7, 8, 9, 10, 20, 30, 40, 50, 60, 70, 80, 90 or 100-fold.

In still another embodiment of the invention, a method of diagnosing a disease associated with CKD is provided comprising the steps of: a) obtaining a biological sample from a subject; b) measuring in the sample the expression level of a plurality of molecular biomarkers selected from the group consisting of those biomarkers identified in Table 5, wherein the biomarkers include the identified mRNA transcription products of the genes identified in Table 5 and proteins encoded thereby; and c) comparing the expression profile of the molecular biomarkers to a control expression profile, thereby diagnosing a disease associated with CKD. In one specific embodiment, the aforementioned is provided wherein the disease associated with CKD is selected from the group consisting of those diseases identified in Table A. In specific embodiments, the aforementioned method is provided wherein the biological sample is a kidney tissue sample or urine sample. In another specific embodiment, the aforementioned method is provided wherein the subject is human. In still another specific embodiment, the aforementioned is provided wherein the expression level of the mRNA transcription products is measured by contacting the biological sample with nucleic acid probes under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, thereby allowing the formation of a hybrid complex between the mRNA transcription products and the nucleic acid probes; and detecting the formation of the complex. In still another specific embodiment, the aforementioned method is provided wherein the level of protein expression is measured using antibodies, wherein at least one antibody is capable of specifically binding to each of the proteins. In another specific embodiment, the aforementioned method is provided wherein the expression of at least one molecular biomarker in the sample is increased relative to the control and the expression of at least one other molecular biomarker in the sample is decreased, relative to the control. In yet another specific embodiment, the aforementioned method is provided wherein a 2- to 10-fold increase or decrease in the amount of expression of the plurality of molecular biomarkers complex compared to a control is indicative of a likelihood of CKD progression. In a related embodiment, the increase or decrease in the amount of expression is 2- to 3, 4, 5, 6, 7, 8, 9, 10, 20, 30, 40, 50, 60, 70, 80, 90 or 100-fold.

In still another embodiment of the invention, a method for assessing the progression of CKD in a subject comprising: a) obtaining a biological sample from a subject; b) measuring in the sample the expression level of a plurality of molecular biomarkers selected from the group consisting of those biomarkers identified in Table 5, wherein the biomarkers include the identified mRNA transcription products of the genes identified in Table 5 and proteins encoded thereby; and c) measuring expression level of the plurality of molecular biomarkers in the sample at a second time point; and d) comparing the expression level in step b) with the expression level in step c), thereby assessing the progression of CKD. In specific embodiments, the aforementioned method is provided wherein the biological sample is a kidney tissue sample or urine sample. In another specific embodiment, the aforementioned method is provided wherein the subject is human. In still another specific embodiment, the aforementioned is provided wherein the expression level of the mRNA transcription products is measured by contacting the biological sample with nucleic acid probes under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, thereby allowing the formation of a hybrid complex between the mRNA transcription products and the nucleic acid probes; and detecting the formation of the complex. In still another specific embodiment, the aforementioned method is provided wherein the level of protein expression is measured using antibodies, wherein at least one antibody is capable of specifically binding to each of the proteins. In another specific embodiment, the aforementioned method is provided wherein the expression of at least one molecular biomarker in the sample is increased relative to the control and the expression of at least one other molecular biomarker in the sample is decreased, relative to the control. In yet another specific embodiment, the aforementioned method is provided wherein a 2- to 10-fold increase or decrease in the amount of expression of the plurality of molecular biomarkers complex compared to a control is indicative of a likelihood of CKD progression. In a related embodiment, the increase or decrease in the amount of expression is 2- to 3, 4, 5, 6, 7, 8, 9, 10, 20, 30, 40, 50, 60, 70, 80, 90 or 100-fold.

In still another embodiment of the invention, a microarray for measuring gene expression characteristic of kidney cells is provided comprising at least 3 polynucleotides encoding a gene or fragment thereof selected from the group consisting of those polynucleotides identified in Table 4. In related embodiments, at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 polynucleotides are contemplated in the aforementioned method.

In another embodiment of the invention, a kit useful for diagnosing a risk of CKD progression is provided comprising: a) at least 3 nucleic acid probes that hybridize under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, to a nucleic acid comprising a sequence selected from the partial or complete coding region sequence of a gene or fragment thereof selected from the group consisting of those polynucleotides identified in Table 4; b) primer pairs useful for PCR-amplifying the nucleic acid sequences in a); and c) instructions for using the probe and primers to facilitate the diagnosis of a risk of CKD progression. In related embodiments, at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 polynucleotides are contemplated in the aforementioned method.

In yet another embodiment, a kit useful for diagnosing a risk of CKD progression is provided comprising at least 3 antibodies, wherein each antibody specifically binds to a unique polypeptide selected from the group consisting of AXL, BGN, COL6A1, CREB3, DKK3, ITGB5, NCF2, S100A6, SLC13A3 and MPV17L; and a reagent useful for the detection of a binding reaction between the antibodies and the polypeptides. In related embodiments, at least 4, 5, 6, 7, 8, 9, or 10 antibodies are contemplated in the aforementioned method. In related embodiments, at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 polypeptide and antibody pairs are contemplated in the aforementioned method.

### BRIEF DESCRIPTION OF THE DRAWINGS

The following drawings form part of the present specification and are included to further illustrate aspects of the present disclosure. The disclosure may be better understood by reference to the drawings in combination with the detailed description of the specific embodiments presented herein.

FIG. 1A shows the bootstrapped hierarchical clustering analysis of 10 wild-type and 18 Tg mice based on expression values of the 43 genes at 4 and 6 weeks of age. Gene expression values were acquired from cDNA microarray data. Transcripts are annotated with their cognate gene symbols. The individual experimental mouse was labeled as “age-Mouse ID.” Black line of the supporting tree indicates the cluster is 100% reproducible.

FIG. 1B shows a scatter plot graph of the histopathological score (evaluated by level of tubular atrophy and interstitial inflammation based on PAS (Periodic Acid-Schiff) staining) of individual mice in groups I, II, and III, as clustered by bootstrapped hierarchical clustering analysis in FIG. 1A. Detailed scoring method is described in Example 1 herein.

FIG. 1C shows the distribution of experimental mice of group I+II and of group III as defined by gene expression profiles in nonprogressor or progressor subgroups as defined by histopathological scores (a mouse is classified as a progressor if the semi-quantitative histopathological score is more than 2, otherwise it is considered to be a nonprogressor, and the nonprogressor subgroup includes wild-type mice).

FIG. 2A shows the bootstrapped hierarchical clustering analysis of uninephrectomized wild-type (n=4) and Tg mice (n=20) at 4 weeks of age based on the expression value of 19 predictive genes in their 2-week-old kidneys. Gene expression values were acquired from 2-week-old kidneys by qrt-PCR. Phenotypic lesions of the remaining kidneys were scored at 4 weeks of age.

FIG. 2B presents the semi-quantitative histopathological score (0 to 4) of the experimental mice in groups I and II (clustered by expression value).

FIG. 2C shows the distribution of experimental mice according to group I or II as defined by gene expression profile and as progressor and nonprogressor by histopathological scores (a mouse is classified as a progressor if the score is more than 2, otherwise it is considered to be a nonprogressor, and the nonprogressor subgroup includes wild-type mice).

FIG. 3A shows a statistical analysis of the functional relationship between tubulointerstitial compartment gene expression and renal function in 50 CKD patients. Ridge regression analysis of tubulointerstitial compartment gene expression values (45 probeset corresponding to 30 human orthologs of the 43 mouse genes) and continuous actual eGFR (estimated glomerular filtration rate; ml/minute/1.73 m²) shows significant relationship with a cross-validated R²=0.53, P<0.001.

FIG. 3B shows the performance of the ridge regression model as a classifier of two groups of CKD patients with measured eGFR higher or lower than 60 l/minute/1.73 m², respectively.

FIG. 4 shows immunohistochemical analyses of protein expression patterns in kidney sections of wild-type and Tg mice with nonprogressive and progressive kidney lesions at 4 weeks of age (n≧5 for each group). FIG. 4A shows single glomerulus staining for Ncf2, S100a6, and Slc13a3. FIG. 4B shows cortex-medulla staining for Mpv171 and Ncf2 with insets of cortex-medulla images. Original magnifications: X63 (A); X10 (B); X40 (B, insets).

FIG. 5 shows immunohistochemical analyses of protein expression patterns in kidney biopsies of patients with various stages of CKD and IgA nephropathy.

FIG. 5A shows a representative glomerular staining for NCF2, S100A6, and SLC13A3. Groups 1 and 2, patients with CKD I/II, GFR value greater than 60 ml/minute/1.73 m²; group 3, patients with CKD III/IV, GFR value is less than 60 ml/minute/1.73 m². FIG. 5B shows tubular staining for NCF2 and BGN. Groups 1, 2, and 3, as described in A. Original magnifications, X40.

### DETAILED DESCRIPTION OF THE PREFERRED EMBODIMENTS OF THE INVENTION

To begin to overcome these challenges to the development of urgently needed predictive markers of CKD progression, the present disclosure provides biomolecular markers for Chronic Kidney Diseases (CKD) and kits thereof, as well as methods of screening for biomolecular markers, methods of diagnosing CKD, and methods of assessing the risk of progression of CKD. In one embodiment of the present disclosure, a two-step comparative genomics approach is used that combines unique resources focused on systematic molecular analysis of murine renal models (Bottinger and Zavadil, Exp. Nephrol., 2002, 10:93-101, incorporated herein by reference) and human kidney biopsy (Cohen, et al., Kidney Int. 2002, 61:133-140, incorporated herein by reference). The initial development of predictive expression signatures for CKD progression was accomplished by applying microarray analysis of whole mouse kidney obtained from the Tgfb1 Tg mouse model, which recapitulates key pathomechanisms and heterogeneity of progression of CKD. This information was then applied to interrogate a human kidney gene expression database to develop and validate a model algorithm for prediction of estimated GFR in humans with various stages of CKD (I to V).

There are five stages of CKD, but kidney function is normal in Stage 1 and minimally reduced in Stage 2. The K/DOQI stages of kidney disease are:

| Stage | GFR* | Description |
|---|---|---|
| 1 | 90+ | Normal kidney function but urine findings or structural |
| | | abnormalities or genetic trait point to kidney disease |
| 2 | 60-89 | Mildly reduced kidney function, and other findings |
| | | (as for stage 1) point to kidney disease |
| 3A | 45-59 | Moderately reduced kidney function |
| 3B | 30-44 | |
| 4 | 15-29 | Severely reduced kidney function |
| 5 | <15 or on | Very severe, or end-stage kidney failure (i.e., |
| | dialysis | established renal failure) |

*All GFR values are normalized to an average surface area (size) of 1.73 m².

Although chronic kidney disease (CKD) is common, only a fraction of CKD patients progress to end-stage renal disease. Molecular predictors to stratify CKD populations according to their risk of progression remained undiscovered until the present disclosure. As described in detail below, the present disclosure reveals the transcriptional profiling of kidneys from Transforming Growth Factor-131 transgenic (Tg) mice, characterized by heterogeneity of kidney disease progression, to identify 43 genes that discriminate kidneys by severity of glomerular apoptosis before the onset of tubulointerstitial fibrosis in 2-week-old animals. Among the genes examined, 19 showed significant correlation between mRNA expression in the excised left kidneys of uninephrectomized Tg mice at 2 weeks of age and renal disease severity in the right kidneys of Tg mice at 4 weeks of age. Gene expression profiles of human orthologs of the 43 genes in kidney biopsies were highly significantly related (R²=0.53; P<0.001) to the estimated glomerular filtration rates in patients with CKD stages I to V, and discriminated groups of CKD stages I/II and III/IV/V with positive and negative predictive values of 0.8 and 0.83, respectively. Protein expression patterns for selected genes were successfully validated by immunohistochemistry in kidneys of Tg mice and kidney biopsies of patients with IgA nephropathy and CKD stages Ito V, respectively. Thus, the present disclosure provides novel mRNA and protein expression signatures that predict progressive renal fibrosis in mice and are useful molecular predictors of CKD progression in humans.

Unless otherwise defined, all technical and scientific terms used herein have the same meaning as commonly understood by one of ordinary skill in the art to which the technology disclosed herein belongs. The following references provide one of skill with a general definition of many of the terms used in this disclosure: Singleton, et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger, et al. (eds.), Springer Verlag (1991); and Hale and Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that, as used in this specification and the appended claims, the singular forms “a,” “an,” and “the” include plural reference unless the context clearly dictates otherwise.

As used herein a “polypeptide” refers to a polymer composed of amino acid residues, structural variants, related naturally occurring structural variants, and synthetic non-naturally occurring analogs thereof linked via peptide bonds. Synthetic polypeptides can be prepared, for example, using an automated polypeptide synthesizer. The term “protein” typically refers to large polypeptides, as well as proteinaceous forms comprising a plurality of polypeptide or peptide chains. The term “peptide” typically refers to short polypeptides.

As used herein a “fragment” of a polypeptide is meant to refer to any portion of a polypeptide or protein smaller than the full-length polypeptide or protein expression product.

A “plurality” refers preferably to a group of at least 5 or more members, more preferably to a group of at least about 9, and even more preferably to a group of at least about 10 members. The maximum number of members is unlimited. The term “gene” or “genes” refers to a polynucleotide sequence(s) of a gene which may be the partial or complete sequence and may comprise regulatory region(s), untranslated region(s), or coding regions. Preferably, a gene comprises a heritable unit containing a coding region for an RNA or protein (or polypeptide), and any associated regulatory region(s). In the present disclosure, the exemplified genes were initially identified from kidney tissue samples.

The term “antibody” is used in the broadest sense and includes fully assembled antibodies, monoclonal antibodies, polyclonal antibodies, multispecific antibodies (e.g., bispecific antibodies), antibody fragments that can bind antigen (e.g., Fab′, F′(ab)₂, Fv, single chain antibodies, diabodies), and recombinant peptides comprising the forgoing as long as they exhibit the desired biological activity.

As used herein, the term “diagnosing” refers to identifying a subject as having a particular disease or disorder or identifying a subject as having a risk of disease or disorder progression based on numerous criteria including, but not limited to, mRNA transcript level, protein expression level, subject symptoms, and/or family history.

As used herein, the phrase “risk of CKD progression” refers to the probability of CKD progression in subjects based on one or more of numerous criteria including, but not limited to, mRNA transcript level, protein expression level, subject symptoms, and/or family history. In some embodiments described herein, the risk of CKD progression is based on either mRNA transcript levels or protein expression levels of a plurality of genes or gene fragments disclosed herein.

As used herein, the term “molecular biomarkers” refers to DNA and/or RNA polynucleotides, including genes and/or mRNA transcripts, and fragments or variants thereof, as well as proteins, polypeptides and/or peptides, and fragments or variants thereof. “Variant” refers to a polynucleotide or polypeptide differing from the polynucleotide or polypeptide of the present disclosure, but retaining essential properties thereof. Generally, variants are overall closely similar, and, in many regions, identical to the polynucleotide or polypeptide of the present disclosure.

In order to conduct sample analysis, a sample containing target polynucleotides is provided. The samples can be any sample containing target molecular biomarkers (e.g., polynucleotides) and can be obtained from any bodily fluid (blood, urine, saliva, phlegm, gastric juices, and the like), cultured cells, biopsies, or other tissue preparations.

DNA or RNA can be isolated from the sample according to any of a number of methods well known to those of skill in the art. For example, methods of purification of nucleic acids are described in Tijssen, Laboratory Techniques in Biochemistry and Molecular Biology: Hybridization With Nucleic Acid Probes, Part I. Theory and Nucleic Acid Preparation, Elsevier, New York N.Y. 1993. In one case, total RNA is isolated using the TRIZOL reagent (Life Technologies, Gaithersburg Md.), and mRNA is isolated using oligo d(T) column chromatography or glass beads. Alternatively, when target polynucleotides are derived from an mRNA, the target polynucleotides can be a cDNA reverse transcribed from an mRNA, an RNA transcribed from that cDNA, a DNA amplified from that cDNA, an RNA transcribed from the amplified DNA, and the like. When the target polynucleotide is derived from DNA, the target polynucleotide can be DNA amplified from DNA or RNA transcribed from DNA. In yet another alternative, the targets are target polynucleotides prepared by more than one method.

When target polynucleotides are amplified, it is desirable to amplify the nucleic acid sample and maintain the relative abundances of the original sample, including low abundance transcripts. Total mRNA can be amplified by reverse transcription using a reverse transcriptase and a primer consisting of oligo d(T) and a sequence encoding the phage T7 promoter to provide a single-stranded DNA template. The second DNA strand is polymerized using a DNA polymerase and a RNAse which assists in breaking up the DNA/RNA hybrid. After synthesis of the double-stranded DNA, T7 RNA polymerase can be added, and RNA transcribed from the second DNA strand template (Van Gelder et al. U.S. Pat. No. 5,545,522). RNA can be amplified in vitro, in situ or in vivo (See Eberwine, U.S. Pat. No. 5,514,545).

Quantitation controls may be included within the sample to ensure that amplification and labeling procedures do not change the true distribution of target polynucleotides in a sample. For this purpose, a sample is spiked with a known amount of at least one control target polynucleotide and the composition of probes includes reference probes that specifically hybridize with the control target polynucleotide(s). After hybridization and processing, the hybridization signals obtained should accurately reveal the amount(s) of control target polynucleotide(s) added to the sample.

Prior to hybridization, it may be desirable to fragment the nucleic acid target polynucleotides. Fragmentation improves hybridization by minimizing secondary structure and cross-hybridization to other nucleic acid target polynucleotides in the sample or to noncomplementary polynucleotide probes. Fragmentation can be performed by mechanical or chemical means known in the art.

The target polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³H, ¹⁴C, ³²P, ³³P or ³⁵S, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Exemplary dyes include quinoline dyes, triarylmethane dyes, phthaleins, azo dyes, cyanine dyes, and the like. Preferably, fluorescent markers absorb light above about 300 nm, preferably above 400 nm, and usually emit light at wavelengths at least greater than 10 nm above the wavelength of the light absorbed. Preferred fluorescent markers include fluorescein, phycoerythrin, rhodamine, lissamine, and C3 and C5 available from Amersham Pharmacia Biotech (Piscataway N.J.).

Labeling can be carried out during an amplification reaction, such as polymerase chain reactions and in vitro transcription reactions, or by nick translation or 5′ or 3′-end-labeling reactions. When the label may be incorporated after or without an amplification step, the label may be incorporated by using terminal transferase or by phosphorylating the 5′ end of the target polynucleotide using, e.g., a kinase and then incubating overnight with a labeled oligonucleotide in the presence of T4 RNA ligase.

Alternatively, the labeling moiety can be incorporated after hybridization once a probe/target complex has formed.

The present disclosure provides, in certain embodiments, methods for assessing gene expression (e.g., amount of mRNA transcripts in a sample). Such methods employ oligonucleotide probes comprising complementary sequences relative to the nucleic acids in the sample. As used herein, the term “complementary sequences” means nucleic acid sequences that are substantially complementary, as may be assessed by the same nucleotide comparison set forth above, or as defined as being capable of hybridizing to a nucleic acid under stringent conditions such as those described herein. Those of skill in the art will understand what is meant by stringent conditions and are referred to page 11.45 of Molecular Cloning: A laboratory Manual, 2nd Ed. Cold Spring Harbor Laboratory Press, Cold Spring Harbor, N.Y., or the conditions set forth in the Summary section of the disclosure, above. Preferred hybridization conditions are stringent hybridization conditions, such as hybridization at 42° C. in a solution (i.e., a hybridization solution) comprising 50% formamide, 1% SDS, 1 M NaCl, 10% dextran sulfate, and washing twice for 30 minutes at 60° C. in a wash solution comprising 0.1×SSC and 1% SDS. It is understood in the art that conditions of equivalent stringency can be achieved through variations of temperature and buffer, or salt concentration, as described in Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage composition of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

As used herein, an oligonucleotide that “specifically hybridizes” to a nucleic acid means that hybridization under suitably (e.g., high) stringent conditions allows discrimination of one or a few hybridizing sequences, preferably one sequence, from other genes. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more nucleotides will be used, although others are contemplated. Longer polynucleotides encoding 250, 500, or 1000 bases and longer are contemplated as well. Such oligonucleotides will find use, for example, as probes in Southern and Northern blots and as primers in amplification reactions.

DNA-based arrays (i.e., “micro-arrays”) provide a simple way to explore the expression of a single polymorphic gene or a large number of genes. In the present disclosure, 15 or more nucleic acids may be presented in a DNA microarray for the analysis of expression of these genes in a sample. Microarray chips are well known to those of skill in the art (see, e.g., U.S. Pat. Nos. 6,308,170; 6,183,698; 6,306,643; 6,297,018; 6,287,850; 6,291,183, each incorporated herein by reference). These are exemplary patents that disclose nucleic acid microarrays and those of skill in the art are aware of numerous other methods and compositions for producing microarrays.

The present disclosure also provides, in certain embodiments, methods for assessing protein expression in a biological sample. Protein expression analyses are well known in the art and include, but are not limited to, Western blots and ELISA, which are described in, for example, Current Protocols in Molecular Biology (1999), Current Protocols in Immunology (2007) John Wiley & Sons, NY, which are incorporated herein by reference in their entireties.

It will be appreciated that the methods of the present disclosure may be useful in fields of human medicine and veterinary medicine. Thus, the “subject” to be treated may be a mammal, preferably human or other animal. For veterinary purposes, subjects include, for example, farm animals such as cows, sheep, pigs, horses and goats, companion animals such as dogs and cats, exotic and/or zoo animals, laboratory animals including mice, rats, rabbits, guinea pigs, and hamsters; and poultry such as chickens, turkey, ducks and geese.

It will also be appreciated from the present disclosure that molecular biomarkers identified in one subject (e.g., a mouse) may be used as a basis to identify orthologous molecular biomarkers in a different subject (e.g., a human).

In one embodiment of the disclosure, the methods described herein are useful for the diagnosis of CKD (and the assessment of risk, or prediction, of CKD progression), as well as diagnosing diseases associated with CKD. “Diseases associated with CKD” include, but are not limited to major causes of chronic kidney diseases as identified in Table A.

TABLE A
Major Causes of Chronic Kidney Disease

Glomerulopathies (primary): Focal glomerulosclerosis, Idiopathic
crescentic glomerulonephritis, IgA nephropathy, Membranoproliferative
glomerulonephritis, Membranous nephropathy
Glomerulopathies associated with systemic disease: Amyloidosis, Diabetes
mellitus, Hemolytic-uremic syndrome, Postinfectious glomerulonephritis,
SLE, Wegener's granulomatosis
Hereditary nephropathies: Hereditary nephritis (Alport's syndrome),
Medullary cystic disease, Nail-patella syndrome, Polycystic kidney disease
Hypertension: Malignant glomerulosclerosis, Nephroangiosclerosis
Obstructive uropathy: Benign prostatic hyperplasia, Posterior urethral
valves, Retroperitoneal fibrosis, Ureteral obstruction (congenital, calculi,
malignancies), Vesicoureteral reflux
Renal macrovascular disease (vasculopathy of renal arteries and veins)
Renal artery stenosis caused by atherosclerosis or fibromuscular dysplasia
Chronic tubulointerstitial nephropathies: Balkan nephropathy,
Immunologic Amyloidosis, Cryoglobulinemia, Goodpasture's syndrome,
IgA nephropathy, Renal transplant rejection, Sarcoidosis, Sjögren's
syndrome, SLE
Cystic diseases: Acquired cystic disease, Medullary cystic disease,
Medullary sponge kidney, Nephronophthisis, Polycystic kidney disease
Drugs Analgesics: Antineoplastics (cisplatin, nitrosourea), Immuno-
suppressives (cyclosporine, tacrolimus), Lithium
Infection Renal parenchymal: Hantavirus—Puumula type (nephropathia
epidemica), Pyelonephritis, Tuberculosis
Mechanical Obstructive uropathy: Reflux nephropathy
Others: Heavy metals Cadmium, Lead, Metabolic Chronic hypokalemia,
Cystinosis, Hypercalcemia, hypercalciuria, Hyperoxaluria, Hyperuricemia,
hyperuricosuria, Hematologic Aplastic anemia, Lymphoma, Multiple
myeloma, Sickle cell anemia, Hereditary nephropathy associated with
hyperuricemia and gout, Radiation nephritis, Vascular Atheroembolism,
Hypertension, Renal vein thrombosis

### EXAMPLES

The following examples are provided for illustration and are not intended to limit the scope of the disclosed subject matter. Briefly, the present disclosure combines unique experimental animal and clinical research resources to identify and characterize molecular markers as predictors of estimated glomerular filtration rate (eGFR) and clinical stage of CKD in human cohorts. First, gene expression profiling was used in cross-sectional and prospective study designs to identify and validate genes that predict heterogeneity in renal disease progression in Transforming Growth Factor-β (TGF-β) Tg mice. In a second step, expression values of human orthologs of these genes, available in a unique database of human kidney biopsies (Schmid, et al., Nephrol. Dial. Transplant 2004, 19:1347-1351 and Yasuda, et al., Clin. Exp. Nephrol. 2006, 10:91-98) were shown to be highly-significantly related to linear GFR or K/DOQI CKD stages in human cohorts affected with hypertensive nephrosclerosis, IgA nephropathy, minimal change disease, or thin basement membrane disease. Finally, immunohistochemical protein expression patterns for several markers were associated with renal lesions in patients with CKD and IgA nephropathy.

Among candidate pathways mediating CKD progression, the TGF-β pathway is prominent because it controls principal pathobiological processes associated with CKD, including fibrogenesis, apoptosis, epithelial-to-mesenchymal transition, and inflammation (Taal and Brenner, Kidney Int. 2006, 70:1694-1705 and Bottinger, Semin. Nephrol, 2007, 27:309-320). Indeed, TGF-β and its receptors are increased in most forms of CKD in humans and experimental animal models (Border and Noble, J. Med. 1994, 331:1286-1292; Bitzer, et al., Kidney Blood Press Res. 1998, 21:1-12; Liu, Y, Kidney Int. 2006, 69:213-217) In addition, DNA polymorphisms of codon 10 in the TGF-β gene have been associated with progressive CKD (Patel, et al., Diabet. Med. 2005, 22:69-73 and August and Suthanthiran, Kidney Int. Suppl. 2003, 87:S99-S104). Consistent with numerous clinical studies, overexpression of TGF-β1 in transgenic (Tg) mice can cause glomerulosclerosis and tubulointerstitial fibrosis (Sanderson, et al., Proc. Natl. Acad. Sci. USA 1995, 92:2572-2576). Similar to the heterogeneity of CKD progression observed in humans, a fraction of TGF-β1 Tg mice develop progressive glomerulosclerosis and tubulointerstitial fibrosis leading to marked proteinuria, uremia, and death, whereas the majority of animals manifest moderate, nonprogressive renal fibrosis with mild proteinuria and normal lifespan (Sanderson, et al., Proc. Natl. Acad. Sci. USA 1995, 92:2572-2576 and Kopp, et al., Lab Invest. 1996, 74:991-1003). The heterogeneity of renal disease manifestations in TGF-β1 Tg mice is dependent on their mixed genetic background, mimicking the familial clustering and importance of genetic susceptibility observed in CKD progression in humans (Satko, et al., Semin. Dial 2007, 20:229-236).

### Example 1

### Materials and Methods

The following materials and methods were used in the experiments described in Examples 2-6.

Mouse Models

Albumin/Tgfb1 Tg mice (Kopp, et al., Lab Invest. 1996, 74:991-1003) in a C57BL/6J X CBA background were maintained at the Animal Resource Center of Mount Sinai School of Medicine. Experiments were performed according to an approved protocol of the institutional animal care and use committee.

Kidney Total RNA Isolation

Harvested mouse kidneys were homogenized in Trizol reagent (Invitrogen, Carlsbad, Calif.) for 40 seconds using PowerGen125 (Fisher Scientific, Pittsburgh, Pa.) at maximum speed. Total RNA was isolated according to the manufacturer's protocol. Quality and quantity of total RNA was checked by Bio-analyzer (Agilent, Santa Clara, Calif.).

Histological Analysis

Harvested kidneys were embedded in paraffin after fixing in 10% formalin overnight and then sectioned at 4-μm thickness. Periodic acid-Schiff (PAS)-stained sections were examined for glomerulosclerosis, mesangial expansion, tubular atrophy, and interstitial inflammation. For in situ detection of macrophage, anti-Mac-3 (BD Biosciences Pharmingen, San Jose, Calif.) was used. Anti-collagen I (Biogenesis, Mill Creek, Wash.) antibody was used to detect collagen accumulation. Apoptotic nuclei were detected by terminal dUTP nick-end labeling (TUNEL) assay (Chemicon, Temecula, Calif.), as described previously (Schiffer, et al., J. Biol. Chem., 2004, 279:37004-37012).

At least 50 glomeruli per kidney were evaluated by two renal pathologists in a blinded manner. Glomerulosclerosis was scored as proportion of glomeruli with sclerosis relative to all glomeruli examined per mouse. Ten X40 fields were scored for tubular atrophy/dilation/casts and for interstitial or perivascular inflammation for each mouse on the following scale: 0=none, 1=<25% of tubules/vessels with tubular features or adjacent mononuclear cells, 3≧50% and 4≧75% of tubules/vessels with those features or with adjacent mononuclear cells. Mean values were used for tubular and interstitial scores.

Other semi-quantitative histopathological scores include: Mac-3 positive cells per tubular interstitial highpower field, Col1a1-positive cells per total glomerular area (%), TUNEL-positive cells were counted as podocytes when residing on the outer aspect of PAS-positive basement membrane. Podocyte apoptotic score was defined as apoptotic podocyte/100 glomeruli. Cells were counted as nonpodocyte glomerular cells when residing inside the outer aspect of PAS-positive basement membrane. Nonpodocyte glomerular apoptotic score was defined as apoptotic nonpodocyte glomerular cells/100 glomeruli. Tubular interstitial apoptotic cell was defined as apoptotic cells/per tubular interstitial high-power field. All of these methods have been previously reported from this laboratory (Bitzer, et al., Genes Dev. 2000, 14:187-197 and Schiffer, et al., J. Clin. Invest. 2001, 108:807-816), and the meanings of these terms and methods for determination are incorporated by reference herein.

Uninephrectomy

Two-week-old mice were anesthetized with isoflurane. After removing hair from the left flank of each mouse, an incision was made and the left kidney was decapsulated, ligated with silk suture, and excised. The area was cleansed with antimicrobial agent Amerse (ConvaTec, St. Louis, Mo.), and the flank incision was sutured closed. Total RNA was isolated from the left kidneys and Quantitative Reverse Transcription Polymerase Chain Reaction (qrt-PCR) was performed for gene expression analysis. At 4 weeks of age, these mice were euthanized and the right kidneys were harvested. One-half of the right kidney was snap-frozen for RNA isolation, and the other half was fixed in 10% normal buffered formalin for histopathological studies.

Quantitative Real-Time (qrt) PCR

One μg of kidney total RNA was reverse-transcribed into single-strand cDNA. The qrt-PCR was performed as described previously (Ju et al., Mol. Cell. Biol., 2006, 26:654-667, incorporated herein by reference). Expression of GAPDH and β-actin were used to normalize the sample amount.

cDNA Microarray

Mouse cDNA arrays (9M series) were obtained from the Albert Einstein College of Medicine cDNA Microarray Facility. Each slide contained an unbiased, random collection of 8976 cDNA probe elements derived from the sequence-verified GEM1 clone set (Incyte Genomics, Palo Alto, Calif.). Microarray procedures were performed as previously described (Yang, et al., Proc. Natl. Acad. Sci. USA, 2003, 100:10269-10274, incorporated herein by reference). For each hybridization, cDNA was prepared from RNA samples obtained from individual kidneys from Tg or Wt mouse (Cy3-labeled) and co-hybridized with a standard reference cDNA prepared from age-matched, pooled RNA obtained from wild-type mouse kidneys (Cy5-labeled).

Data Processing of Murine Data

A. Preprocessing and Normalization

In the preprocessing step, the signal intensities were not background-subtracted because of the local nature in which background intensity was calculated. All spots flagged during the scanning and quantification steps were removed from further analysis. The array had a scarcity of negative controls preventing an is-expressed threshold from being set. The data were normalized using a within-slide scaled loess normalization that corrects for print-tip effects.

B. Tg (Progressive) versus Tg (Non-Progressive) Comparisons

The strength of the correlation (using Spearman coefficients) among phenotypic traits in the Tg group was used to determine informative traits. The Tg mice were then clustered using hierarchical sampling based on a phenotypic similarity metric for podocyte apoptosis. Rank order statistics and permutation tests were used to compare expression patterns among the different Tg animals. Finally, biclustering was done to identify subsets of genes and samples that when one is used to cluster the other, stable and significant partitions emerge. This allows utilization of both the phenotypic and expression matrices.

Clustering Analysis

Hierarchical cluster dendrograms were generated with TIGR Multiexperiment Viewer software (The Institute for Genomics Research, Rockville, Md.) by using Manhattan distance metrics and bootstrapping protocols for resampling.

Immunohistochemistry Staining

Immunohistochemistry staining was performed using Vectastain ABC systems (Vector Laboratories, Burlingame, Calif.) and anti-Ncf2 (Santa Cruz Biotechnology Inc., Santa Cruz, Calif.), anti-Itgb5 (Abcam, Cambridge, Mass.), anti-Bgn (Abcam), anti-Col6a1 (Santa Cruz), anti-S100a6 (Santa Cruz), anti-Dkk3 (Santa Cruz), anti-Slc13a3 (Abcam), and anti-Mpv171 (affinity-purified in our laboratory (Krick et al., Proc. Natl. Acad. Sci. USA, 2008, 105:14106-14111)) antibodies. Images were generated by a Zeiss (Thornwood, N.Y.) Axioskop microscope in Mount Sinai Medical Center.

Gene Expression Analysis of Human Renal Biopsies

A. Microdissection and RNA Isolation

After renal biopsy, the tissue was transferred to RNase inhibitor and microdissected into glomerular and tubular fragments. Total RNA was isolated from microdissected tubulointerstitial and glomerular tissue as previously described (Cohen and Kretzler, Nephron., 2002, 92:522-528, incorporated herein by reference).

B. Target Preparation

A total of 300 to 800 ng of total RNA was reverse-transcribed and linearly amplified according to a protocol previously reported (Schmid et al., Diabetes, 2006, 55:2993-3003, incorporated herein by reference). The fragmentation, hybridization, staining, and imaging were performed according to the Affymetrix (Santa Clara, Calif.) Expression Analysis Technical Manual.

Image files were initially obtained through Affymetrix GeneChip software. Subsequently, robust multichip analysis was performed using RMAexpress. Robust multichip analysis is an R-based technique using the Affymetrix microarray image file and is comprised of three steps: background adjustment, quartile normalization, and summarization. The expression values for the Affymetrix probesets are reported as log2 transformed.

Affymetrix-based gene expression profiling was performed as described in detail (Schmid et al., Diabetes, 2006, 55:2993-3003, incorporated herein by reference). In brief, human renal biopsy specimens were procured in an international multicenter study, the European Renal cDNA Bank-Kroener-Fresenius biopsy bank. Biopsies were obtained from patients after informed consent and with approval of the local ethics committees.

C. Regression and Prediction of GFR at Time of Biopsy

Ridge regression was chosen to improve the predictive potential of the model in the presence of collinearities among the markers (Hoerl and Kennard, Technometrics, 2007, 12:55-67, incorporated herein by reference). Additionally the Pearson correlation between the expression values and the MDRD GFR of the patient for each probeset was calculated. To estimate the significance of the correlation coefficients, the false discovery rate was determined using a permutation approach (Benjamini and Hochberg Y, 1995, Series B (Methodological), 57 (1), 289-300).

### Example 2

### Identification of a Gene Expression Signature of Advanced Glomerular Apoptosis Activity in Kidneys of Tgfb1 Tg Mice

Kidneys from 2-week-old Tg mice were characterized by severe podocyte apoptosis and mild mesangial expansion in some, but not all, animals, while the tubulointerstitial compartment was normal (Schiffer, et al., J. Clin. Invest. 2001, 108:807-816). To identify gene expression patterns that are associated with quantitative measures of apoptosis, extracellular matrix accumulation, or inflammatory cell infiltrates at the early stage of progressive renal disease in this model, respectively, microarray and detailed quantitative phenotype analyses in wild-type and Tgfb1 Tg mice were performed. Matrix accumulation and tubulointerstitial inflammation were assessed by quantitative digital analysis of α-1-collagen 1 and Mac3 immunohistochemistry, respectively. Glomerular and tubulointerstitial cell apoptosis rates were quantitated by TUNEL assay. With the exception of glomerular apoptosis rates, the quantitative phenotype markers were either not significantly different among Tg mice (glomerular anti-Col1a1 labeling), and/or not significantly different when compared with wild-type mice (tubulointerstitial apoptosis and anti-Mac3 labeling) (Table 1).

TABLE 1
Quantitative Analysis of Extracellular Matrix Accumulation, Inflammation,
and Apoptosis in Wild-Type (Wt) and TGF-β1 Transgenic (Tg) Mice.

| Parameters | Tg6 | Tg5 | Tg2 | Tg3 | Tg7 | Tg1 | Tg4 | Wt1 | Wt2 | Wt3 | Wt4 | Wt5 |
|---|---|---|---|---|---|---|---|---|---|---|---|---|
| Col1a1 | 0.20 | 0.22 | 0.34 | 0.21 | 0.37 | 0.25 | 0.20 | 0.00 | 0.00 | 0.00 | 0.00 | 0.00 |
| Mac-3 | 0.00 | 0.00 | 2.00 | 1.00 | 1.00 | 0.00 | 1.00 | 0.00 | 0.00 | 0.56 | 0.56 | 0.56 |
| GA | 0.00 | 2.22 | 4.77 | 10.00 | 13.16 | 16.67 | 22.73 | 0.00 | 2.03 | 0.06 | 0.91 | 0.00 |
| TIA | 1.60 | 1.42 | 1.40 | 1.15 | 3.05 | 2.67 | 1.09 | 0.71 | 0.61 | 1.21 | 0.48 | 0.22 |

Col1a1, collagen I α-1-positive area, presented as fraction of Col1a1-positive area per total glomerular area; Mac-3, presented as Mac-3-positive cells per high-power field; GA, glomerular cell apoptosis was scored as apoptotic glomerular cells/100 glomeruli; TIA, tubular interstitial apoptosis was defined as apoptotic cells/per tubular interstitial high-power field based on TUNEL assay.

In contrast, glomerular apoptosis rates (GA) were sufficiently variable to separate Tgfb1 Tg mice into two groups as defined by GA less than threefold (Tg2, Tg5, Tg6), or more than threefold (Tg1, Tg3, Tg4, Tg7) of maximum value observed in wild-type control mice, respectively (Table 1). To identify the genes that are differentially expressed in the two groups, linear discriminant analyses (Brabender, et al., Oncogene 2004, 23:4780-4788) were performed on the expression patterns of 9000 genes. Linear discriminant analysis is a statistical method usually used to find the linear combination of features that best separate two or more classes of objects or events. Linear discriminant analysis revealed 43 genes with significantly different expression patterns between Tg mice with GA less than threefold and more than threefold of wild-type control. Quantitative assays for these 43 genes (Table 2) were developed to quantitate their mRNA levels in three additional studies and to evaluate their use as molecular classifiers and/or predictors of progressive renal disease.

TABLE 2
43 murine genes that discriminate high versus low glomerular apoptosis rates in
2-wk-old TGF-β1 transgenic mice: Entrez Gene ID (Mus musculus), Gene Symbol
(Mus musculus), Official Full Gene Name (Mus musculus), Human Ortholog Gene
Symbol (Homo Sapiens), and Human Ortholog Entrez Gene ID (Homo sapiens).

| Entrez Gene ID | Gene Symbol | | Gene Symbol | Entrez Gene ID |
|---|---|---|---|---|
| (Mm) | (Mm) | Official Full Name (Mm) | (Hs) | (Hs) |
| 280662 | Afm | Afamin | Afm | 173 |
| 23796 | Agtrl1 | Angiotensin receptor-like 1 | Agtrl1 | 429337 |
| 30878 | Apln | Apelin | Apln | 8862 |
| 26362 | Axl | AXL receptor tyrosine kinase | Axl | 558 |
| 12010 | B2m | Beta-2 microglobulin | B2m | 567 |
| 12111 | Bgn | Biglycan | Bgn | 633 |
| 12514 | Cd68 | CD68 antigen | Cd68 | 968 |
| 93694 | Clec2d | C-type lectin domain family 2, | Clec2d | 29121 |
| | | member d | | |
| 12833 | Col6a1 | Procollagen, type VI, alpha 1 | Col6a1 | 1291 |
| 56264 | Cpxm1 | Carboxypeptidase X1 (M14 family) | Cpsm1 | 56265 |
| 12913 | Creb3 | cAMP responsive element binding | Creb3 | 10488 |
| | | protein 3 | | |
| 13032 | Ctsc | Cathepsin C | Ctsc | 1075 |
| 20312 | Cx3cl1 | Chemokine (C-X3-C motif) ligand 1 | Cx3cl1 | 6376 |
| 18214 | Ddr2 | Discoidin domain receptor family, | Ddr2 | 4921 |
| | | member 2 | | |
| 50781 | Dkk3 | Dickkopf homolog 3 (Xenopus laevis) | Dkk3 | 27122 |
| 94176 | Dock2 | Dedicator of cyto-kinesis 2 | Dock2 | 1794 |
| 22240 | Dpysl3 | Dihydropyrimidinase-like 3 | Dpysl3 | 1809 |
| 207521 | Dtx4 | Deltex 4 homolog (Drosophila) | Dtx4 | 23220 |
| 18301 | Fxyd5 | FXYD domain-containing ion | Fxyd5 | 53827 |
| | | transport regulator 5 | | |
| 54393 | Gabbr1 | Gamma-aminobutyric acid (GABA-B) | Gabbr1 | 2550 |
| | | receptor, 1 | | |
| 14469 | Gpb2 | Guanylate nucleotide binding protein 2 | Gpb2 | 2634 |
| 14860 | Gsta4 | Glutathione S-transferase, alpha 4 | Gsta4 | 2941 |
| 14904 | Gtpbp1 | GTP binding protein 1 | Gtpbp1 | 9567 |
| 14960 | H2-Aa | Histocompatibility 2, class II antigen | HLA-DQA1 | 3117 |
| | | A, alpha | | |
| 14998 | H2-DMa | Histocompatibility 2, class II, locus | HLA-DMA | 3108 |
| | | DMa | | |
| 50786 | Hs6st2 | Heparin sulfate 6-O-sulfotransferase 2 | Hs6st2 | 90161 |
| 15904 | Id4 | Inhibitor of DNA binding 4 | Id4 | 3400 |
| 16009 | Igfbp3 | Insulin-like growth factor binding | Igfbp3 | 282261 |
| | | protein 3 | | |
| 16419 | Itgb5 | Integrin beta 5 | Itgb5 | 3693 |
| 16483 | Kap | Kidney androgen regulated protein | Not found | Data not found |
| 16819 | Lcn2 | Lipocalin 2 | Lcn2 | 3934 |
| 93734 | Mpv17I | Mpv17 transgene, kidney disease | Mpv17I | 255027 |
| | | mutant-like | | |
| 17970 | Ncf2 | Neutrophil cytosolic factor 2 | Ncf2 | 4688 |
| 76477 | Pcolce2 | Procollagen C-endopeptidase enhancer 2 | Pcolce2 | 26577 |
| 235587 | Parp3 | Poly (ADP-ribose) polymerase family, | Parp3 | 10039 |
| | | member 3 | | |
| 20200 | S100a6 | S100 calcium binding protein A6 | S100a6 | 6277 |
| | | (calcyclin) | | |
| 114644 | Slc13a3 | Solute carrier family 13 (sodium- | Slc13a3 | 64849 |
| | | dependent dicarboxylate transp.), | | |
| | | member 3 | | |
| 20564 | Slit3 | Slit homolog 3 (Drosophila) | Slit3 | 6586 |
| 64074 | Smoc2 | SPARC related modular calcium | Smoc2 | 64094 |
| | | binding 2 | | |
| 66442 | Spc25 | Spindle pole body component 25 | Spc25 | 57405 |
| | | homolog (S. cerevisiae) | | |
| 21858 | Timp2 | Tissue inhibitor of metalloproteinase 2 | Timp2 | 7077 |
| 24099 | Tnfsf13b | Tumor necrosis factor (ligand) | Tnfsf13b | 10673 |
| | | superfamily, member 13b | | |
| 22436 | Xdh | Xanthine dehydrogenase | Xdh | 7498 |

### Example 3

### Validation of 43-Gene Expression Signature to Classify Tubulointerstitial Disease Heterogeneity in Older Tgfb1 Tg Mice

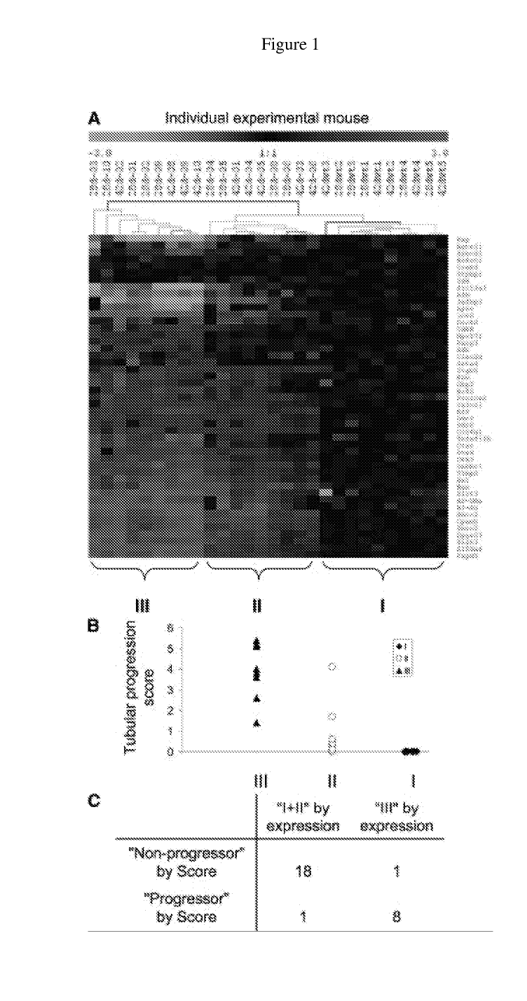

By 4 or 6 weeks of age, histopathological manifestations of progressive renal disease, including tubular atrophy and interstitial inflammation, were established and highly variable in Tgfb1 Tg mice. Next, the question of whether the expression patterns of the 43-geneset (i.e., set of 43 genes described in Example 2) in 4- and 6-week-old mice are suitable for classifying the mice by progression of histopathological manifestations in the kidney was addressed. An interrelated two-way (that is, genes against mice) hierarchical clustering method was implemented using an unsupervised approach. The goal of clustering is to find important gene expression patterns and perform cluster discovery on experimental mice. The advantage of this approach is that the relationships between the groups of genes and mice can be used dynamically while iteratively clustering through both gene dimension and experimental mouse dimension. Unsupervised two-way hierarchical clustering was applied, based on expression profiles of the 43-geneset, to an independent set of 4- and 6-week-old wild-type (10 mice) and Tgfb1 Tg (18 mice). To show the reliability of the clusters, bootstrapped cluster analysis (the bootstrap is widely accepted as a method to assess the reliability of reconstructed phylogenetic trees (Kerr and Churchill, Proc. Natl. Acad. Sci. USA, 2001, 98:8961-8965) was performed using the TMEV (TIGR Multiple Experiment Viewer) program (Saeed et al., Biotechniques, 2003, 34:374-378) and it was demonstrated that expression profiles of 43-geneset reliably identified a highly-significant (100% reproducibility) cluster of 9 animals (cluster III) among 18 Tgfb1 Tg mice and 10 wildtype mice (FIG. 1A).

Semi-quantitative scores for tubular atrophy (TA) and interstitial-perivascular inflammation (IPI) were tightly correlated in 4- to 6-week-old Tgfb1 Tg mice and combined to calculate a composite tubular progression score (TA+IPI) in all animals of this set. The composite tubular progression score was significantly higher in cluster III (median, 4.0) compared with cluster II Tgfb1 Tg mice (median, 0.4) (P=0.00015) and cluster I (wild-type mice) (FIG. 1B). A cut-off value of 2.0 of the composite tubular progression score classified Tgfb1 Tg and wild-type mice into progressive (cluster III) and nonprogressive (clusters I, II) groups with 88.9% and 95% sensitivity and specificity, respectively (FIG. 1C). These findings indicate that the expression profiles of the 43-geneset discriminated, and thereby classified, advanced versus mild tubulointerstitial progression of renal disease with high sensitivity and specificity in an independent set of older Tgfb1 Tg mice.

### Example 4

### Identification of Prospective Predictor Expression Signatures for Progressive Renal Fibrosis in Tgfb1 Tg Mice

A longitudinal study was devised to examine the correlation and predictive values of gene expression profiles obtained among the 43-geneset in left kidneys removed by uninephrectomy at 2 weeks of age, with the histopathological manifestations in right kidneys of the same animal at 4 weeks of age. Expression levels of the 43-geneset were determined by qrt-PCR analysis of total RNA extracted from whole kidney of 24 experimental mice (20 Tgfb1 Tg mice and 4 wild-type mice) at 2 weeks of age. Histopathological scoring of the remaining right kidney was performed on PAS-stained sections by three independent investigators in a blinded manner at 4 weeks of age. F-test statistics identified 19 genes (Table 3) among the 43-geneset, which grouped animals prospectively according to the severity of histopathological scores with statistical significance of P<0.05.

TABLE 3
19 murine genes that grouped animals prospectively according to the severity of
histopathological scores: Entrez Gene ID (Mus musculus), Gene Symbol (Mus musculus),
Official Full Gene Name (Mus musculus), Human Ortholog Gene Symbol (Homo Sapiens),
and Human Ortholog Entrez Gene ID (Homo sapiens).

| Entrez Gene ID | Gene Symbol | | Gene Symbol | Entrez Gene ID |
|---|---|---|---|---|
| (Mm) | (Mm) | Official Full Name (Mm) | (Hs) | (Hs) |
| 30878 | Apln | Apelin | Apln | 8862 |
| 26362 | Axl | AXL receptor tyrosine kinase | Axl | 558 |
| 12111 | Bgn | Biglycan | Bgn | 633 |
| 12833 | Col6a1 | Procollagen, type VI, alpha 1 | Col6a1 | 1291 |
| 56264 | Cpxm1 | Carboxypeptidase X 1 (M14 | Cpsm1 | 56265 |
| | | family) | | |
| 12913 | Creb3 | cAMP responsive element | Creb3 | 10488 |
| | | binding protein 3 | | |
| 13032 | Ctsc | Cathepsin C | Ctsc | 1075 |
| 50781 | Dkk3 | dickkopf homolog 3 (Xenopus | Dkk3 | 27122 |
| | | laevis) | | |
| 207521 | Dtx4 | Deltex 4 homolog (Drosophila) | Dtx4 | 23220 |
| 18301 | Fxyd5 | FXYD domain-containing ion | Fxyd5 | 53827 |
| | | transport regulator 5 | | |
| 16419 | Itgb5 | Integrin beta 5 | Itgb5 | 3693 |
| 93734 | Mpv17l | Mpv17 transgene, kidney | Mpv17l | 255027 |
| | | disease mutant-like | | |
| 17970 | Ncf2 | Neutrophil cytosolic factor 2 | Ncf2 | 4688 |
| 235587 | Parp3 | Poly (ADP-ribose) polymerase | Parp3 | 10039 |
| | | family, member 3 | | |
| 20200 | S100a6 | S100 calcium binding protein | S100a6 | 6277 |
| | | A6 (calcyclin) | | |
| 114644 | Slc13a3 | Solute carrier family 13 | Slc13a3 | 64849 |
| | | (sodium-dependent | | |
| | | dicarboxylate transp.), member 3 | | |
| 64074 | Smoc2 | SPARC related modular | Smoc2 | 64094 |
| | | calcium binding 2 | | |
| 66442 | Spc25 | Spindle pole body component | Spc25 | 57405 |
| | | 25 homolog (S. cerevisiae) | | |
| 24099 | Tnfsf13b | Tumor necrosis factor (Ligand) | Tnfsf13b | 10673 |
| | | superfamily, member 13b | | |

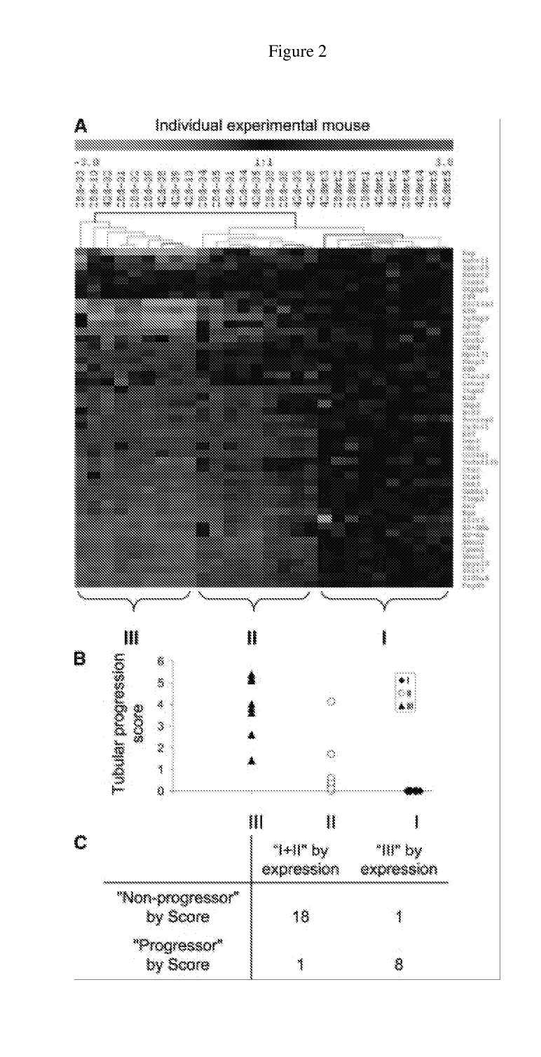

Bootstrapped unsupervised clustering demonstrated that expression profiles of the 19-geneset, determined by qrt-PCR of left kidney RNA at 2 weeks of age, reliably identified a highly-significant (100% reproducibility) cluster of 10 animals (cluster II) among 20 Tgfb1 Tg mice and 4 wild-type mice (FIG. 2A). Histopathology scores were recorded for each animal on a scale of 0 (normal) to 4 (global glomerulosclerosis and tubulointerstitial fibrosis). Scores were consistent across all three investigators and the median score for each animal was used for statistical analysis. The median of all median histopathology scores among animals grouped in cluster II (Tgfb1 Tg mice) was 3.0, compared with 0 among all animals in cluster I (10 Tgfb1 Tg and 4 wild-type mice) (P=0.000078) (FIG. 2B). By assigning an optimal cut-off value of the histopathology score between 1 and 2 for classification, the gene expression profiles for the 19-geneset, as determined at 2 weeks of age, prospectively predicted the severity of renal disease progression as assessed by semi-quantitative scoring in Tgfb1 Tg mice with 87.5%, 88.9%, and 86.6% accuracy, sensitivity, and specificity, respectively (FIG. 2C). Thus, results obtained from the prospective, longitudinal validation study in an independent cohort of Tgfb1 Tg and wild-type mice demonstrated that the 19-geneset predicted advanced versus mild progression of renal disease with high accuracy, sensitivity, and specificity. Of note, renal TGF-β1 mRNA levels were not significantly different between the advanced and mild progression groups identified by the 19-geneset expression signature, indicating that the separation of mild and advanced progression groups is not associated with differences in renal TGF-β1 levels between both groups.

### Example 5

### Development of a Renal Gene Expression Signature Related to Continuous eGFR and CKD Stages in Human CKD Cohorts

Because the 43-geneset and a subset of 19 genes were validated as classifier and/or predictive molecular biomarkers of advanced renal histopathology in two independent cohorts of Tgfb1 Tg mice using a cross-sectional and a longitudinal study design, respectively, a third validation study was devised using cohorts of humans with various stages of CKD from the European Renal cDNA Bank-Kroener-Fresenius Biopsy Bank (ERCB) (Schmid et al., Nephrol. Dial. Transplant, 2004, 19:1347-1351). Consented ERCB participants were screened for cases with i) diagnostic kidney biopsies; ii) quality-controlled, high-quality RNA/cDNA from microdissected tubular interstitial and glomerular compartments; iii) high-quality genome-wide expression microarray data for both, glomerular and tubulointerstitial compartments; and iv) clinical information on medical treatments and K/DOQI stage classification of kidney function. This screen of the ERCB database identified patients with hypertensive nephropathy (HTN) (n=19), IgA nephropathy (IgAN) (n=21), minimal change disease (n=1), thin membrane disease (n=6), and unaffected, normal renal tissue from tumor nephrectomies (n=3).

Analysis of gene expression data, ascertained by Affymetrix HGU133A GeneChip analysis applied to the tubulointerstitial compartment samples, revealed that 45 probesets, interrogating 30 human orthologs among the 43 murine geneset, genes, passed quality control thresholds forminimum expression values[Note: A probe set on Affymetrix GeneChips is a collection of probes designed to interrogate a given sequence. A probe set name is used to refer to a probe set, which looks like the following: 12345_at or 12345_a_at or 12345_s_at or 12345_x_at].

TABLE 4
Probe set ID, Gene Name, and Gene Symbols for 30 human orthologs
with significant gene expression levels in tubuointerstial
compartment samples from human kidney biopsies.

| | | Gene |
|---|---|---|
| Probesets | Gene Name | Symbol |
| 206840_at | afamin | AFM |
| 202686_s_at | AXL receptor tyrosine kinase | AXL |
| 202685_s_at | AXL receptor tyrosine kinase | AXL |
| 216231_s_at | beta-2-microglobulin | B2M |
| 201891_s_at | beta-2-microglobulin | B2M |
| 201261_x_at | biglycan | BGN |
| 213905_x_at | biglycan /// teashirt family zinc finger 1 | BGN |
| 203507_at | CD68 molecule | CD68 |
| 212937_s_at | collagen, type VI, alpha 1 | COL6A1 |
| 213428_s_at | collagen, type VI, alpha 1 | COL6A1 |
| 212938_at | collagen, type VI, alpha 1 | COL6A1 |
| 212091_s_at | collagen, type VI, alpha 1 | COL6A1 |
| 209432_s_at | cAMP responsive element binding protein 3 | CREB3 |
| 201487_at | cathepsin C | CTSC |
| 823_at | chemokine (C-X3-C motif) ligand 1 | CX3CL1 |
| 203687_at | chemokine (C-X3-C motif) ligand 1 | CX3CL1 |
| 205168_at | discoidin domain receptor family, member 2 | DDR2 |
| 214247_s_at | dickkopf homolog 3 (Xenopus laevis) | DKK3 |
| 201431_s_at | dihydropyrimidinase-like 3 | DPYSL3 |
| 212611_at | deltex 4 homolog (Drosophila) | DTX4 |
| 218084_x_at | FXYD domain containing ion transport | FXYD5 |
| | regulator 5 | |
| 203146_s_at | gamma-aminobutyric acid (GABA) B | GABBR1 |
| | receptor, 1 | |
| 202748_at | guanylate binding protein 2, interferon- | GBP2 |
| | inducible /// guanylate binding protein 2, | |
| | interferon-inducible | |
| 202967_at | glutathione S-transferase A4 | GSTA4 |
| 205276_s_at | GTP binding protein 1 | GTPBP1 |
| 205274_at | GTP binding protein 1 | GTPBP1 |
| 219357_at | GTP binding protein 1 | GTPBP1 |
| 209293_x_at | inhibitor of DNA binding 4, dominant | ID4 |
| | negative helix-loop-helix protein | |
| 209291_at | inhibitor of DNA binding 4, dominant | ID4 |
| | negative helix-loop-helix protein | |
| 209292_at | Inhibitor of DNA binding 4, dominant | ID4 |
| | negative helix-loop-helix protein | |
| 210095_s_at | insulin-like growth factor binding protein 3 | IGFBP3 |
| 212143_s_at | insulin-like growth factor binding protein 3 | IGFBP3 |
| 201125_s_at | integrin, beta 5 | ITGB5 |
| 214020_x_at | Integrin, beta 5 | ITGB5 |
| 201124_at | integrin, beta 5 | ITGB5 |
| 212531_at | lipocalin 2 (oncogene 24p3) | LCN2 |
| 209949_at | neutrophil cytosolic factor 2 (65 kDa, | NCF2 |
| | chronic granulomatous disease, autosomal 2) | |
| 209940_at | poly (ADP-ribose) polymerase family, | PARP3 |
| | member 3 | |
| 219295_s_at | procollagen C-endopeptidase enhancer 2 | PCOLCE2 |
| 217728_at | S100 calcium binding protein A6 | S100A6 |
| 205244_s_at | solute carrier family 13 (sodium-dependent | SLC13A3 |
| | dicarboxylate transporter), member 3 | |
| 205243_at | solute carrier family 13 (sodium-dependent | SLC13A3 |
| | dicarboxylate transporter), member 3 | |
| 203813_s_at | slit homolog 3 (Drosophila) | SLIT3 |
| 203167_at | TIMP metallopeptidase inhibitor 2 | TIMP2 |
| 210301_at | xanthine dehydrogenase | XDH |

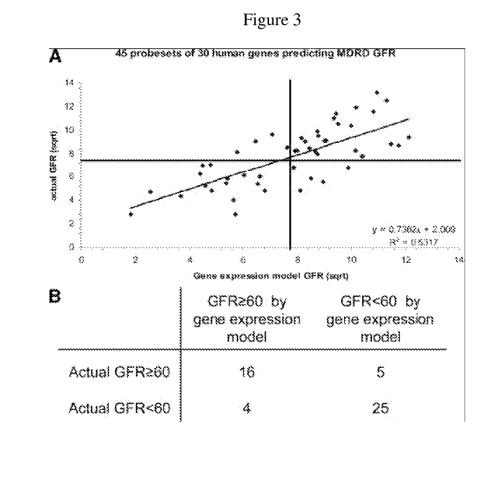

Although the severity of progressive renal disease is typically assessed in murine models by histopathological, but not functional, parameters, stages of human CKD are well-defined and assessed by measured or eGFR (Snyder and Pendergraph, Am. Fam. Physician, 2005, 72:1723-1732, incorporated herein by reference). The eGFR at time of kidney biopsy was calculated using a modified MDRD formula for all patients eligible for this study (Levey et al., Ann Intern. Med., 1999, 130:461-470). eGFR ranged from 7.58 ml/minute/1.73 m2 to 157.30 ml/minute/1.73 m2 across the entire cohort, including tumor nephrectomy patients (n=3) and patients with stage I (n=8), stage II (n=18), stage III (n=11), stage 1V (n=8), and stage V (n=2) CDK as defined by K/DOQI CKD staging criteria (Am. J. Kidney Dis., 2002, 39:S1-S266; Snyder and Pendergraph, Am. Fam. Physician, 2005, 72:1723-1732; Bauer et al., J. Am. Soc. Nephrol., 2008, 19:844-846, all three publications incorporated herein by reference). To test whether the continuous actual eGFR is statistically related to the expression values of the ortholog geneset in glomerular and/or tubular interstitial compartments, regression analysis was applied using a ridge regression model and leave-one-out cross-validation as previously described (Hoerl and Kennard, Technometrics, 2007, 12:55-67 and Bovelstad et al., Bioinformatics, 2007, 23:2080-2087, each incorporated herein by reference). The glomerular expression dataset did not achieve statistically significant relationship results of the actual MDRD eGFR. In contrast, the tubular interstitial expression levels for 30 human orthologs assessed by 45 probesets (Table 4) provided a highly-significant relationship of continuous actual eGFR with a cross-validated R2 of 0.53 (r=0.74, P<0.001) (FIG. 3A). Clinical cohort studies demonstrate that patients with CKD stages III, IV, and V (GFR<60 ml/minute/1.73 m2) have a high likelihood of progressive CKD (Taal and Brenner, Kidney Int. 2006, 70:1694-1705). In contrast, progression of CKD in patients with CKD stages I and II (GFR>60 ml/minute/1.73 m2) remains poorly defined. By assigning the clinically relevant eGFR threshold of 60 ml/minute/1.73 m2 between stages II and III as a cutoff, the tubulointerstitial expression signatures of the 45 human probeset-classified patients into stage I/II or stage III/IV/V groups was effected with 83%, 80%, 86.2%, and 76.2% positive predictive value (PPV), negative predictive value (NPV), sensitivity, and specificity, respectively (FIG. 3B).

### Example 6

### Comparative Characterization of Marker Protein Expression Patterns in Kidneys of Tgfb1 Tg Mice and Patients with IgA Nephropathy

Individual tubular expression profiles for 16 genes (Table 5) were significantly correlated with continuous actual eGFR by univariate statistical analysis (false discovery rate<0.01).

TABLE 5
16 human genes whose tubulo-interstitial mRNA
level correlates with the MDRD GFR of the patients
by single probe evaluation (FDR < 0.01)

| Probe Set ID | FDR | Correlation Coefficient | Gene Symbol |
|---|---|---|---|
| 206840_at | 0 | 0.69 | AFM |
| 202686_s_at | 0 | −0.46 | AXL |
| 216231_s_at | 0 | −0.5 | B2M |
| 201261_x_at | 0 | −0.49 | BGN |
| 212937_s_at | 0 | 0.56 | COL6A1 |
| 209432_s_at | 0 | −0.37 | CREB3 |
| 205168_at | 0 | 0.38 | DDR2 |
| 214247_s_at | 0 | −0.63 | DKK3 |
| 203146_s_at | 0 | 0.5 | GABBR1 |
| 202748_at | 0 | −0.66 | GBP2 |
| 201125_s_at | 0 | −0.72 | ITGB5 |
| 209949_at | 0 | −0.44 | NCF2 |
| 217728_at | 0 | −0.69 | S100A6 |
| 205243_at | 0 | 0.57 | SLC13A3 |
| 203813_s_at | 0 | −0.35 | SLIT3 |
| 210301_at | 0 | 0.42 | XDH |

Because RNA profiling of diagnostic kidney biopsies is currently limited to research applications, studies were initiated to examine the protein expression profiles of gene products from these 16 genes in Tgfb1 Tg mice. Antibodies were obtained from commercial or academic sources where available. Their utility in immunohistochemistry of kidney sections from wild-type and Tgfb1 Tg mice with moderate (histopathology score 1) or advanced (score 3) renal disease at 4 weeks of age was evaluated for specificity and quality, providing satisfactory results for the following antibodies: neutrophil cytosolic factor 2 (Ncf2), biglycan (Bgn), integrin β5 (Itgb5), collagen type VI, α 1 (Col6A1), 5100 calcium-binding protein A6 (S100a6), solute carrier family 13 (sodium-dependent dicarboxylate transporter), member 3 (Slc13a3) and dickkopf 3 (Dkk3). In addition, a recently generated antibody for Mpv17-like protein (Mpv171), was used (Krick, et al., Proc. Natl. Acad. Sci. USA 2008, 105:14106-14111, incorporated herein by reference). Results are summarized in Table 6.

TABLE 6
Protein Expression Signatures in Glomeruli (Glom.), Tubules
(Tub.), and Interstitial Cells (Int.) as Assessed by Immunolabeling
of Sections from Wild-Type (Wt) Mice and Tg Mice with
Nonprogressive or Progressive Kidney Disease
(n ≧ Five Animals per Group)

| | WT | | | Nonprogressive | | | Progressive | | |
|---|---|---|---|---|---|---|---|---|---|
| Protein | Glom. | Tub. | Int. | Glom. | Tub. | Int. | Glom. | Tub. | Int. |
| Ncf2 | − | − | − | − | − | − | ++ | ++ | − |
| Mpv17I | − | ++ | − | − | + | − | − | − | − |
| S100a6 | − | − | − | − | − | + | + | − | ++ |
| Bgn | − | +/− | − | + | ++ | − | ++ | ++ | − |
| Col6A1 | − | + | − | − | ++ | − | − | ++ | − |
| Itgb5 | + | + | − | ++ | ++ | − | ++ | ++ | − |
| Slc13a3 | − | + | − | − | + | − | + | + | − |
| Dkk3 | − | − | − | + | − | − | ++ | − | − |

Staining for Ncf2, a 67-kDa cytosolic subunit of the multiprotein complex of NADPH oxidase in neutrophils, was modest and comparable in tubules of wild-type and score 1 Tgf1b Tg mice, but strongly increased in tubules of score 3 Tgfb1 Tg mice, where de novo expression was also observed in glomerular cells, representing a podocyte-like pattern. Biglycan is a member of the small leucine repeat proteoglycan family (SLRP) that is barely detectable in tubules of wild-type kidney, but strongly increased in tubules, parietal epithelial cells, and glomerular cells with a podocyte pattern in score 1, and especially in score 3 Tgfb1 Tg mice. Itgb5 is strongly expressed in kidneys from wild-type mice at the corticomedullary junction and moderately expressed in glomerular cells, whereas it is expressed with increasing intensity in cortical tubules and glomerular cells with a podocyte pattern of score 1 and score 3 Tgfb1 Tg mice. Col6a1 was expressed in tubules of wild-type mice and expression became increased in tubules of Tgfb1 Tg mice and intensity is correlated with disease severity. Mpv171 is a member of the Mpv17/PMP22 protein family that was strongly expressed in tubuli at the cortical and corticomedullary junction tubuli in kidneys of wild-type mice. Cortical tubular expression was greatly reduced in score 1 Tgf1 Tg mice, and both cortical and corticomedullary tubular expression was lost in score 3 Tgfb1 Tg mice.

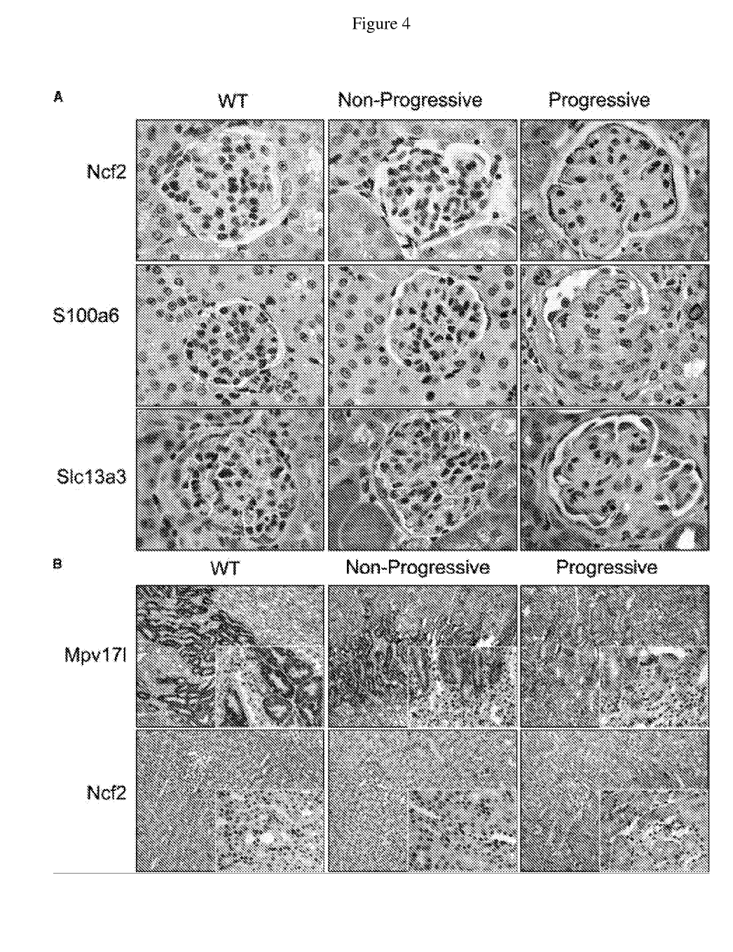

S100a6, also called calcyclin, is a 10.5-kDa calcium-binding protein that is not detectable in kidneys of wild-type mice. S100a6 was detected in cortical interstitial cells in score 1 and score 3 Tgfb1 Tg mice, and in podocytes of score 3 Tgfb1 Tg animals. Slc13a3 is one of the Nat⁺-dependent dicarboxylate transporters that were encoded by Slc13 gene family members. It is highly expressed in cortical and corticomedullary junction tubuli in kidneys of WT and Tgf1 Tg mice. High-level expression of Slc13a3 was also observed exclusively in glomerular cells with a podocyte pattern in score 3 Tgfb1 Tg mice. De novo expression of Dkk3 proteins was detected in podocytes of Tgfb1 Tg mice with nonprogressive disease by double-immunofluorescence staining with podocyte-specific marker synaptopodin. The expression of Dkk3 was further significantly increased in kidneys with progressive kidney disease. Taken together, these observations delineate novel marker protein (and mRNA) expression patterns that distinguish normal kidneys and kidneys with nonprogressive and progressive disease, including de novo podocyte expression of Bgn and Dkk3, de novo cortical interstitial S100a6 expression, and a gradual increase in tubular Bgn, Col6a1, and Itgb5. In contrast, de novo expression of S100a6, Ncf2, and Slc13a3 in glomerular cells with a podocyte pattern (FIG. 4A) and global loss of tubular Mpv171 expression (FIG. 4B) was a characteristic protein expression pattern that distinguished advanced kidney disease from nonprogressive kidney disease in Tgfb1 Tg mice.

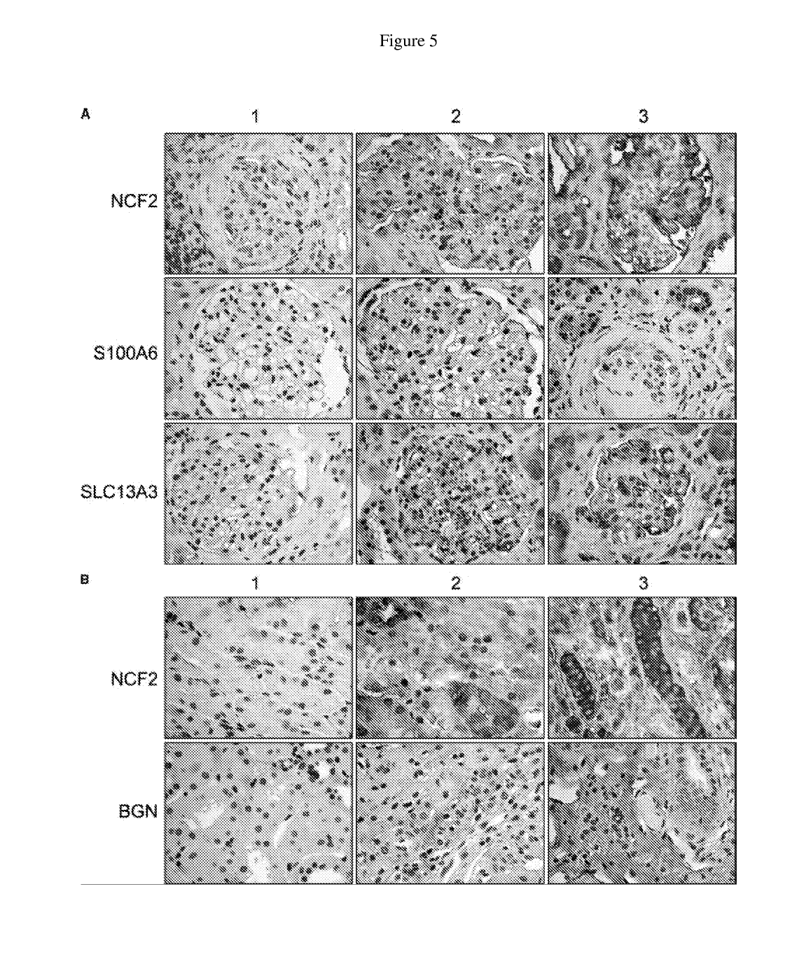

Because no additional tissue was available for confirmatory immunohistochemistry from the ERCB renal biopsy tissue used for expression profiling studies, sections of routine clinical biopsies were obtained from 20 patients with IgA nephropathy and eGFRs between 7 to 134 ml/minute/1.73 m². Four of the eight antibodies evaluated in Tgfb1 Tg mice provided specific and consistent staining patterns when applied to human kidney biopsy sections (FIG. 5). Protein expression patterns of S100A6, NCF2, SLC13A3, and BGN were strongly increased in kidney biopsies of patients with stage III/IV CKD (FIG. 5, A and B; group 3), compared with patients with stage I/II CKD (FIG. 5, A and B; groups 1 and 2), and similar to the expression patterns detected in Tgfb1 Tg mice (FIG. 4A). Interestingly, in the biopsies from patients with stage I/II CKD (eGFR higher 60 ml/minute/1.73 m2), staining for all of these proteins was either present or absent, i.e., these four proteins appeared closely co-expressed.

### Discussion

Although the classification of progressive CKD in the Examples herein was based on distinct endpoints and methods, namely advanced histopathological scores in mouse versus estimated GFR on a continuum of stage I to V in human cohorts, the results of the comparative analysis were remarkably consistent, supporting the validity and robustness of this approach. For example, the intersection between the 19 murine gene expression profiles that were significantly correlated with advanced histopathology scores in Tgfb1 Tg mice, and the 16 human gene expression profiles that were significantly correlated with eGFR in humans, were comprised of nine genes, including Axl, Bgn, Col6a1, Creb3, Dkk3, Itgb5, Ncf2, S100a6, and Slc13a3. Only 4 of 19 murine expression profiles, Ctsc, Dtx4, Fxyd5, and Adprt13 were correlated with progressive disease in our longitudinal validation study in Tgfb1 Tg mice, but were not correlated with eGFR in human CKD, whereas Apin, Cpxml, Mpv171, Smoc2, Spc25, and Tnfsf13b were correlated in mice, but expression data for their human orthologs was not available in the human gene expression database (either the probe sets were not present in Affymetrix HGU133A or the data did not pass quality control as described above). Because previous reports demonstrated that the deletion of the Mpv17 gene, a close homolog of Mpv171, caused nephrotic syndrome and progressive glomerulosclerosis in mice (Weiher et al., Cell, 1990, 62:425-434) qrt-PCR was used to confirm the correlation of MPV17L expression with CKD progression independently. Thus, 10 molecular markers were correlated with CKD progression based on histopathological and functional (eGFR) parameters in murine and human kidney disease, respectively. Among these, only S100a6 (calcyclin) has previously been reported as potential marker for acute ischemic tubular injury (Lewington et al., Am. J. Physiol., 1997, 273:F380-F385 and Cheng et al., Kidney Int., 2005, 68:2694-2703)

The present findings also indicate that unknown pathways play an important role in the imbalances in stress response and cell survival/cell death signaling in CKD progression. Several of the 10 genes have previously been linked with multiple path-ways controlling apoptosis, inflammation, and organization of extracellular matrix, including inducers and modulators of receptor for advanced glycation end products (RAGE), TGF-β, and Akt signaling (S100a6, Bgn, Axl, Dkk3), endoplasmatic reticulum (ER) stress (Creb3), mitochondrial dysfunction (Col6a1, Mpv171), innate immune response (Bgn), and fibrillar collagen network formation (Bgn). For example, Axl is a receptor tyrosine kinase and activator of Akt survival signaling that has previously been implicated in early diabetic nephropathy and experimental glomerulonephritis models, respectively (Yanagita et al., J. Clin. Invest., 2002, 110:239-246 and Nagai et al., Kidney Int., 2005, 68:552-561), whereas S100a6 can modulate cell survival by interacting with distinct RAGE immunglobulin domains (Leclerc et al., J. Biol. Chem., 2007, 282:31317-31331). S100a6 is also involved in the processing of apoptosis by modulating the transcriptional regulation of caspase-3 (Joo et al., J. Cell. Biochem., 2008, 103:1183-1197). Dkk3 may regulate TGF-β signaling in Xenopus (Pinho and Niehrs, Differentiation 2007, 75:957-967), and its overexpression in cancer cells induced apoptosis (Yue et al., Carcinogenesis, 2008, 29:84-92). Mpv171 is an inner mitochondrial membrane protein of proximal tubular cells that protects mitochondria against superoxide generation, apoptosis, and mitochondrial dysfunction (Krick et al., Proc. Natl. Acad. Sci. USA, 2008, 105:14106-14111). Supramolecular assemblies of collagen VI microfibrils provide scaffolds for the formation of the structurally critical fibrillar collagen networks through connection with the small proteoglycans decorin and biglycan (Lampe and Bushby, J. Med. Genet., 2005, 42:673-685). Although biglycan itself functions as an extracellular matrix organizer, it can modulate stress signaling directly by binding extracellular TGF-β, or toll-like receptor 4 (TLR4), which has recently been implicated as innate immune response mediator in hepatic fibrosis and ischemia reperfusion injury models (Seki et al., Nat. Med., 2007, 13:1324-1332 and Wu et al., J. Clin. Invest., 2007, 117:2847-2859).

The leucine zipper transcription factor Creb3 regulates transcription of mediators of endoplasmic reticulum (ER) stress response (Liang et al., Mol. Cell. Biol., 2006, 26:7999-8010). Only the Slc13a3 gene belongs to the family of renal sodium-dicarboxylate co-transporters involved in the handling of citrate by the kidney and has not been implicated in cell stress or apoptosis to date. Together, the functional roles in their respective pathways of the CKD progression gene and protein profiles reported herein support the emerging concept of glomerular and tubular epithelial cell injury and apoptosis as initiating mechanisms in TGF-β-mediated nephron loss and renal fibrosis, considered central pathomechanisms in progression of CKD (Bottinger, Semin. Nephrol., 2007, 27:309-320 and Bottinger, Laboratory Press, 2008, pp 989-1022). Finally, these results are also consistent with the validity of the initial screening strategy of selecting gene expression profiles for development of markers of CKD progression based on their ability to discriminate the extent of epithelial apoptosis in kidneys of Tgfb1 Tg mice before the emergence of histopathological lesions. In routine diagnostic kidney biopsy procedures, typically two tissue cores are obtained by needle biopsy and processed for histopathology, immunofluorescence, or electron microscopy methods. However, microdissection or fractionation of glomerular and tubular tissue compartments, prerequisite steps for standardized, quantitative molecular analysis, are currently only performed in research settings, but not in routine diagnostic biopsy procedures.

To enhance the potential clinical utility of this study, the feasibility of using routine immunodetection methods on kidney sections by interrogating the in situ protein expression profiles of the 10 putative markers of CKD progression that were identified in mouse and human kidney disease was verified. From these, a panel of eight informative molecular markers for CKD progression (Table 2) was developed, whereas antibodies for Axl and Creb3, were either not available, or did not provide specific protein detection, respectively. As characteristic protein expression signatures of advanced disease, de novo expression of Ncf2, S100a6, and Slc13a3 proteins was exclusively detected in glomerular cells with a podocyte pattern of Tgfb1 Tg mice with progressive disease (FIG. 4A), in addition to de novo expression of Ncf2 and significant loss of Mpv171 protein expression in corticomedullary tubules (FIG. 4B). In contrast, de novo expression of Bgn and Dkk3 proteins in glomerular cells with a podocyte pattern, and of Itgb5 in subcortical tubules, was already detected in Tgfb1 Tg mice with nonprogressive disease, and their expression was markedly increased in kidneys manifesting progressive disease compared with nonprogressive disease. Thus, protein expression signatures were developed on the basis of gene expression signatures that distinguish progressive and nonprogressive kidney disease in Tgfb1 Tg mice. In addition, observations with immunohistochemistry on human kidney biopsies indicate that a subset of these protein expression signatures, including S100A6, SLC13A3, BGN, and NCF2 may be applied to archival and prospective human kidney biopsy collections obtained through routine diagnostic biopsy protocols. However, further clinical development and validation of the protein expression signatures for CKD progression identified here will require longitudinal studies of extended cohorts of human CKD patients with diagnostic kidney biopsies that are beyond the scope of our current study and will require collaborative study networks involving multiple centers.

All of the compositions and/or methods disclosed and claimed herein can be made and executed without undue experimentation in light of the present disclosure. While the compositions and methods of this disclosure have been described in terms of specific embodiments, it will be apparent to those of skill in the art that variations of the compositions and/or methods and in the steps or in the sequence of steps of the method described herein can be made without departing from the concept, spirit and scope of the disclosed subject matter. More specifically, it will be apparent that certain agents which are both chemically and physiologically related may be substituted for the agents described herein while the same or similar results are achieved. All such similar substitutes and modifications apparent to those skilled in the art are deemed to be within the spirit, scope and concept of the disclosed subject matter as defined by the appended claims.

The references cited herein throughout, to the extent that they provide exemplary procedural or other details supplementary to those set forth herein, are all specifically incorporated herein by reference.

What is claimed is:

1. A method of assessing the risk of chronic kidney disease (CKD) progression comprising the steps of:
a) obtaining a biological sample from a subject; and
b) measuring in said sample the expression level of a plurality of molecular biomarkers selected from the group consisting of those biomarkers identified in Table 5, wherein the biomarkers include the identified mRNA transcription products of the genes identified in Table 5 and proteins encoded thereby; and
c) comparing the expression profile of said molecular biomarkers to a control expression profile, thereby assessing the risk of CKD progression.

2. The method according to claim 1 wherein the biological sample is a kidney tissue sample.

3. The method according to claim 1 wherein the biological sample is a urine sample.

4. The method according to claim 1 wherein the subject is human.

5. The method according to claim 1 wherein the expression level of said mRNA transcription products is measured by contacting the biological sample with nucleic acid probes under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, thereby allowing the formation of a hybrid complex between the mRNA transcription products and the nucleic acid probes; and detecting the formation of the complex.

6. The method according to claim 1 wherein the level of protein expression is measured using antibodies, wherein at least one antibody is capable of specifically binding to each of the proteins.

7. The method according to claim 1, wherein the expression of at least one molecular biomarker in the sample is increased relative to the control and the expression of at least one other molecular biomarker in the sample is decreased, relative to the control.

8. The method according to claim 1, wherein a 2- to 10-fold increase or decrease in the amount of expression of said plurality of molecular biomarkers complex compared to a control is indicative of a likelihood of CKD progression.

9. A method of screening for a molecular biomarker useful in assessing the risk of CKD progression comprising the steps of:
a) obtaining a biological sample from a subject suffering from CKD or a disease associated with CKD;
b) measuring expression level of a candidate molecular biomarker in said sample; and
c) comparing the expression profile of said candidate molecular biomarker to a control expression profile of said candidate molecular biomarker, thereby identifying a candidate molecular biomarker as a molecular biomarker useful in assessing the risk of CKD progression.

10. The method according to claim 9 wherein the biological sample is a kidney tissue sample.

11. The method according to claim 9 wherein the biological sample is a urine sample.

12. The method according to claim 9 wherein the subject is human.

13. The method according to claim 9 wherein the expression level of said candidate molecular biomarker is measured by contacting the biological sample with a nucleic acid probe under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, thereby allowing the formation of a hybrid complex between the mRNA transcription product and the nucleic acid probe; and detecting the formation of the complex.

14. The method according to claim 9 wherein the level of expression of the candidate molecular biomarker is measured at the protein level by using at least one antibody that specifically binds to the candidate molecular biomarker protein.

15. The method according to claim 9, wherein a 2 to 10-fold increase or decrease in the amount of expression of said candidate molecular biomarker compared to a control is indicative of a risk of CKD progression.

16. A method of diagnosing a disease associated with CKD comprising the steps of:
a) obtaining a biological sample from a subject;
b) measuring in said sample the expression level of a plurality of molecular biomarkers selected from the group consisting of those biomarkers identified in Table 5, wherein the biomarkers include the identified mRNA transcription products of the genes identified in Table 5 and proteins encoded thereby; and
c) comparing the expression profile of said molecular biomarkers to a control expression profile, thereby diagnosing a disease associated with CKD.

17. The method according to claim 16 wherein the disease associated with CKD is selected from the group consisting of those diseases identified in Table A.

18. The method according to claim 16 wherein the biological sample is a kidney tissue sample.

19. The method according to claim 16 wherein the biological sample is a urine sample.

20. The method according to claim 16 wherein the subject is human.

21. The method according to claim 16 wherein the expression level of said mRNA transcription products is measured by contacting the biological sample with nucleic acid probes under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, thereby allowing the formation of a hybrid complex between the mRNA transcription products and the nucleic acid probes; and detecting the formation of the complex.

22. The method according to claim 16 wherein the level of protein expression is measured using antibodies, wherein at least one antibody is capable of specifically binding to each of the proteins.

23. The method according to claim 16, wherein a 2 to 10-fold increase or decrease in the amount of expression of said plurality of molecular biomarkers complex compared to a control is indicative of a risk of CKD progression.

24. A method for assessing the progression of CKD in a subject comprising
a) obtaining a biological sample from a subject;
b) measuring in said sample the expression level of a plurality of molecular biomarkers selected from the group consisting of those biomarkers identified in Table 5, wherein the biomarkers include the identified mRNA transcription products of the genes identified in Table 5 and proteins encoded thereby; and
c) measuring expression level of the plurality of molecular biomarkers in said sample at a second time point; and
d) comparing the expression level in step b) with the expression level in step c), thereby assessing the progression of CKD.

25. The method according to claim 24 wherein the biological sample is a kidney tissue sample.

26. The method according to claim 24 wherein the biological sample is a urine sample.

27. The method according to claim 24 wherein the subject is human.

28. The method according to claim 24 wherein the expression level of said mRNA transcription products is measured by contacting the biological sample with nucleic acid probes under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, thereby allowing the formation of a hybrid complex between the mRNA transcription products and the nucleic acid probes; and detecting the formation of the complex.

29. The method according to claim 24 wherein the level of protein expression is measured using antibodies, wherein at least one antibody specifically binds to each of the proteins.

30. The method according to claim 24, wherein a 2 to 10-fold increase or decrease in the amount of expression of said plurality of molecular biomarkers complex compared to a control is indicative of a risk of CKD progression.

31. A microarray for measuring gene expression characteristic of kidney cells comprising at least 3 polynucleotides encoding a gene or fragment thereof selected from the group consisting of those polynucleotides identified in Table 4.

32. A kit useful for diagnosing a risk of CKD progression comprising:
a) at least 3 nucleic acid probes that hybridize under stringent conditions comprising hybridization at 42° C. in a solution comprising 50% formamide, 1% SDS, 1 M NaCl, 10% dextran sulfate, and washing twice for 30 minutes at 60° C. in a wash solution comprising 0.1×SSC and 1% SDS, to a nucleic acid comprising a sequence selected from the partial or complete coding region sequence of a gene or fragment thereof selected from the group consisting of those polynucleotides identified in Table 4;
b) primer pairs useful for PCR-amplifying the nucleic acid sequences in a); and
c) instructions for using the probe and primers to facilitate the diagnosis of a risk of CKD progression.

33. A kit useful for diagnosing a risk of CKD progression comprising at least 3 antibodies, wherein each antibody specifically binds to a unique polypeptide selected from the group consisting of AXL, BGN, COL6A1, CREB3, DKK3, ITGB5, NCF2, S100A6, SLC13A3 and MPV17L; and a reagent useful for the detection of a binding reaction between said antibodies and said polypeptides.
